(19) United States
(12) Patent Application Publication
(10) Pub. No.: US 2020/0215148 A1
(43) Pub. Date: Jul. 9, 2020

(54) METHOD FOR PRODUCING COMPLEX CONTAINING PROTEIN FOR MEDICAL USE AND POLYAMINO ACID, AND COMPLEX CONTAINING PROTEIN FOR MEDICAL USE AND POLYAMINO ACID

(71) Applicant: TERUMO KABUSHIKI KAISHA, Shibuya-ku, Tokyo (JP)

(72) Inventors: Masahiro MIMURA, Tsukuba-shi, Ibaraki (JP); Kentaro SHIRAKI, Tsukuba-shi, Ibaraki (JP); Ruriko IIBUCHI, Kanagawa (JP); Naoki AKATSUKA, Kanagawa (JP)

(73) Assignee: TERUMO KABUSHIKI KAISHA, Shibuya-ku, Tokyo (JP)

(21) Appl. No.: 16/633,350

(22) PCT Filed: Jul. 24, 2018
(86) PCT No.: PCT/JP2018/027771

§ 371 Date: Mar. 25, 2020

(30) Foreign Application Priority Data
Jul. 24, 2017 (JP) 2017-142905

(51) Int. Cl. A61K 38/02; A61K 47/10; A61K 47/36; A61K 47/42; A61K 47/64

(52) U.S. Cl. CPC A61K 38/02; A61K 47/10; A61K 9/14; A61K 47/42; A61K 47/64; A61K 47/36

(57) ABSTRACT

Provided is a method for producing a protein for medical use/polyamino acid complex, the method being able to more stably improve a formation rate (yield) of the protein for medical use/polyamino acid complex.

A method for producing a protein for medical use/polyamino acid complex, the method including forming the complex in a mixed solution obtained by mixing a solution A containing a polyamino acid and at least one of alcohol or a hydrophilic polymer with a solution B containing a protein for medical use.

### TECHNICAL FIELD

The present invention relates to a method for producing a complex containing a protein for medical use and a polyamino acid, and a complex containing a protein for medical use and a polyamino acid.

### BACKGROUND ART

In accordance with the advance of genetic recombination techniques, various protein drugs have been developed since the 1980s. In particular, the development of antibodies known as molecular target drugs has remarkably contributed to treatments of intractable diseases such as cancer, articular rheumatism, and the like which have been difficult to be treated hitherto.

Unlike low molecular compound drugs, since it is difficult for protein drugs to be orally administered, the protein drugs are often administered into the body by injection. In particular, hypodermic injection has been expected as a new administration method because it causes less pain and can be simply self-administered (self-injection by a patient). However, since a dose of the hypodermic injection is limited to 1.5 mL or less, it is usually required to prepare a highly concentrated protein solution of 100 mg/mL or more.

In order to increase a concentration of a protein drug, a method of redissolving a powder preparation has been widely used. Lyophilized protein powder is merely dissolved in a small amount of solution, but there are some difficulties in practice. First, it should be considered that a protein is unstable. When a protein in powder form is dissolved, a physicochemical stress such as a shear stress, a surface tension, or the like is applied, and the protein may thus be irreversibly denatured. Furthermore, the denatured protein is more likely to be aggregated as a concentration of a protein solution to be prepared is high. Aggregates may not only reduce the efficacy of the protein drug, but also adversely affect safety; for example, unwanted immune reactions and the like may be caused. In addition to such instability of protein, the time required for dissolution is also problematic in practice. In a case where a highly concentrated salt solution is prepared, dissolution can be performed by performing stirring with a stirrer or a vortex, but it is required for a protein to be handled carefully so as not to be denatured due to its instability. In the case of an actual preparation, a solvent such as a physiological saline or the like is added to a powder preparation in a vial, and then the dissolution is performed by slowly shaking the vial so as not to foam. Therefore, several tens of minutes to several hours may be required to dissolve the entire protein powder.

Another approach is to concentrate a protein. That is, there is a method in which the amount of solvent is reduced by selectively removing the solvent from a protein solution with a low concentration to prepare a highly concentrated protein solution. Examples of a typical concentration method include an ultrafiltration method, chromatography, and an evaporation method. Examples of a method of redissolving a powder preparation may include a lyophilization method, a spray dry method, and the like.

However, since the number of operation steps increases, there remain problems such as equipment and time costs. In recent years, a new concentration method that do not require an apparatus, such as liquid-liquid phase separation, gelation, crystallization, or the like has been developed, but there remain problems such as an irreversible denaturation of a protein caused by a treatment, and the like. Therefore, in order to obtain a highly concentrated protein solution, it is expected to develop a method that 1) does not cause denaturation of a protein, 2) includes a simple and quick step, and 3) does not require investment such as an apparatus or the like. In addition, in consideration of an increase in the number of types of protein drugs in the future, it is preferable to use 4) a general method that can be used for various types of proteins without using an antibody, an enzyme, a hormone, or the like.

For example, WO 2015/064591 A discloses a protein for medical use/polyamino acid complex-containing aqueous suspension. The protein for medical use/polyamino acid complex-containing aqueous suspension can be concentrated by removing a solvent, and can be used as a drug by adding an electrolyte with a low concentration to dissociate the protein.

### SUMMARY OF INVENTION

Certainly, WO 2015/064591 A discloses that a protein for medical use/polyamino acid complex-containing aqueous suspension can be concentrated by removing a solvent, and can be used as a drug by adding an electrolyte with a low concentration to dissociate the protein.

However, it may be difficult to form a protein for medical use/polyamino acid complex depending on conditions such as a pH, the type of buffer solution, and the like. Therefore, it is required to more stably improve a formation rate (yield) of a protein for medical use/polyamino acid complex.

Accordingly, an object of the present invention is to provide a method for producing a protein for medical use/polyamino acid complex, the method being able to more stably improve a formation rate (yield) of the protein for medical use/polyamino acid complex.

The present inventors have conducted intensive studies in order to solve the above-described problems. As a result, the present inventors found that the above-described problems are solved by a method for producing a protein for medical use/polyamino acid complex, the method including forming the complex in a mixed solution obtained by mixing a solution A containing a polyamino acid and at least one of alcohol or a hydrophilic polymer with a solution B containing a protein for medical use, thereby completing the present invention.

### BRIEF DESCRIPTION OF DRAWINGS

FIG. 1-1 shows a result of a far ultraviolet CD spectrum of polyE in each of solutions A1 to A5 and A′1 prepared in Test Example 1.

FIG. 1-2 shows a formation rate of an IgG/polyE complex in each of Examples 1-1 to 1-15 and Comparative Example 1.

FIG. 2-1 shows the number of particles per 1 mL of an IgG/polyE complex formed in Test Example 2.

FIG. 2-2 shows a ratio of the number of particles of an IgG/polyE complex formed in each of Examples 2-1 to 2-4 to the number of particles having each particle size of an IgG/polyE complex formed in Comparative Example 2-1, for each particle size.

FIG. 2-3 shows an image of particles of an IgG/polyE complex formed in each of Example 2-4 and Comparative Example 2-1 and an appearance of a mixed solution obtained after the complex is formed.

FIG. 3 shows an IgG concentration (left axis: ▪ and •) and an IgG yield after redissolution (right axis: and ▪) in a case where an IgG/polyE complex precipitate is redissolved with a 150 to 900 mM NaCl-10 mM citrate buffer solution (pH 7.0), in which ▪ and □ indicate a result in the presence of ethanol and • and ∘ indicate a result in the absence of ethanol.

FIG. 4 shows a result of a far ultraviolet CD spectrum of IgG in a wavelength of 200 to 250 nm in Test Example 4.

FIG. 5 shows a relationship between the number of times of washing operations and a recovery rate of IgG from an IgG/polyE complex redissolved in Test Example 5.

FIG. 6-1 shows a result of a far ultraviolet CD spectrum of polyE in each of solutions A17 to A21 and A′1 prepared in Test Example 6.

FIG. 6-2 shows a formation rate of an IgG/polyE complex in each of Examples 6-1 to 6-5 and Comparative Example 6.

FIG. 7 shows a result of a far ultraviolet CD spectrum of IgG in a wavelength of 200 to 250 nm in Test Example 7.

FIG. 8 shows a recovery rate of IgG from an IgG/polyE/PEG complex redissolved in Test Example 8.

FIG. 9 shows a formation rate of an IgG/polyE/PEG or IgG/polyE complex in Test Example 9.

FIG. 10 shows a formation rate of an IgG/polyE/PEG or IgG/polyE complex in Test Example 10.

### DESCRIPTION OF EMBODIMENTS

Hereinafter, an embodiment according to an aspect of the present invention will be described. The present invention is not limited to only the following embodiments.

Herein, “X to Y” representing a range means “X or more and Y or less”. In addition, unless otherwise specified, an operation and a measurement of physical properties and the like are carried out under a condition of room temperature (20 to 25° C.)/relative humidity of 40 to 50% RH.

Herein, a protein for medical use/polyamino acid complex is a complex formed by a protein for medical use and a polyamino acid described later, and the complex may also include components other than the protein for medical use and the polyamino acid, for example, a hydrophilic polymer and the like.

### <<Production Method of Protein for Medical Use/Polyamino Acid Complex>>

An aspect of the present invention is a method for producing a protein for medical use/polyamino acid complex, the method including forming the complex in a mixed solution obtained by mixing a solution A containing a polyamino acid and at least one of alcohol (excluding a hydrophilic polymer) or a hydrophilic polymer (excluding a polyamino acid) with a solution B containing a protein for medical use. With such a configuration, a protein for medical use/polyamino acid complex can be more stably formed, and a formation rate (yield) of the complex can thus be improved.

A preferred embodiment of the present aspect is a production method of a protein for medical use/polyamino acid complex, the production method including forming the complex in a mixed solution obtained by mixing a solution A containing a polyamino acid and alcohol with a solution B containing a protein for medical use. The solution A contains alcohol, that is, the mixed solution contains alcohol, such that a particle size (equivalent circle diameter) of the protein for medical use/polyamino acid complex can be increased, in addition to the above effect of the present invention. In addition, in the case of the protein for medical use/polyamino acid complex, it can be expected that the protein for medical use is rapidly released at the time of administration to a patient and the like. Further, a sustained-release time can be controlled by adjusting a concentration of the alcohol in the mixed solution. Therefore, the protein for medical use/polyamino acid complex produced in the present embodiment is suitable for a drug (an anti-viral antibody or the like) intended to rapidly increase a blood concentration.

A preferred embodiment of the present aspect is a production method of a protein for medical use/polyamino acid complex, the production method including forming the complex in a mixed solution obtained by mixing a solution A containing a polyamino acid and a hydrophilic polymer (excluding a polyamino acid) with a solution B containing a protein for medical use. The solution A contains a hydrophilic polymer, that is, the mixed solution contains a hydrophilic polymer, such that a sustained-release effect of the protein for medical use at the time of administration to a patient and the like can be expected, and a sustained-release time can be controlled by adjusting a concentration of the hydrophilic polymer in the mixed solution, in addition to the above effect of the present invention. Therefore, the protein for medical use/polyamino acid complex produced in the present embodiment is suitable for the case where it is desired to reduce the number of administrations (frequent administrations of hormone preparations and the like), the case where a drug (an antibody for an anti-cancer drug or the like) is desired to locally act, and the like.

### <Solution A>

In the production method of the present aspect, the solution A contains a polyamino acid and at least one of alcohol or a hydrophilic polymer.

### [Polyamino Acid]

In the production method of the present aspect, the protein for medical use/polyamino acid complex can be formed mainly by an electrostatic interaction. Therefore, it is preferable to appropriately select a polyamino acid having a charge opposite to that of a protein for medical use to be used.

Examples of an anionic polyamino acid may include a polyglutamic acid (mass: 750 to 5000 Da, pI: 2.81 to 3.46), a polyglutamic acid (mass: 3000 to 15000 Da, pI: 2.36 to 3.00), a polyglutamic acid (mass: 15000 to 50000 Da, pI: 1.85 to 2.36), a polyglutamic acid (mass: 50000 to 100000 Da, pI: 1.56 to 1.85), a polyaspartic acid (mass: 2000 to 11000 Da, pI: 2.06 to 2.75), a polyaspartic acid (mass: 5000 to 15000 Da, pI: 1.93 to 2.39), water-soluble salts thereof, and the like.

Examples of a cationic polyamino acid may include polylysine (mass: 1000 to 5000 Da, pI: 10.85 to 11.58), polylysine (mass: 4000 to 15000 Da, pI: 11.49 to 12.06), polylysine (mass: 15000 to 30000 Da, pI: 12.06 to 12.37), polylysine (mass: more than or equal to 30000 Da, pI: more than or equal to 12.37), polyarginine (mass: 5000 to 15000 Da, pI: 13.49 to 13.97), polyarginine (mass: 15000 to 70000 Da, pI: 13.98 to 14.00), polyarginine (mass: more than or equal to 70000 Da, pI: 14.00), polyhistidine (mass: 5000 to 25000 Da, pI: 7.74 to 8.30), water-soluble salts thereof, and the like.

Among them, the polyamino acid is preferably selected from the group consisting of a polyglutamic acid, polylysine, polyarginine, and water-soluble salts thereof, from the viewpoint that such polyamino acid has biocompatibility, and a pI at which the polyamino acid easily forms a complex with a protein for medical use, and the like.

The polyamino acid can be used alone or in a combination of two or more thereof, depending on a protein for medical use to be used. It should be noted that the polyamino acid herein excludes a hydrophilic polymer described later.

### [Alcohol]

In the production method of the present aspect, alcohol contained in the solution A is not particularly limited, but, for example, ethanol, methanol, trifluoroethanol (TFE), and the like can be used. The alcohol can be used alone or in a combination of two or more thereof. It should be noted that the alcohol herein excludes a hydrophilic polymer described later.

Alcohol is preferably selected from the group consisting of ethanol, methanol, and TFE, and ethanol is more preferable.

In the production method of the aspect, a mechanism for improving a yield (formation rate) of a protein for medical use/polyamino acid complex by using alcohol is estimated as follows.

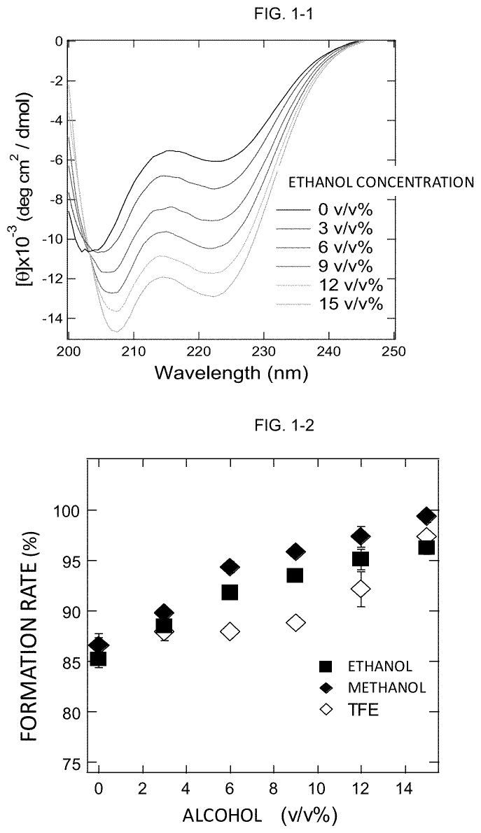

It is considered that a polyamino acid and alcohol are present in a solution, such that a structure of the polyamino acid is changed by the alcohol. Specifically, it is considered that since the alcohol has an effect of decreasing a dielectric constant of the solution, hydrogen bonds forming a three-dimensional structure of the polyamino acid are strengthened, and a secondary structure of the polyamino acid is changed to an a-helix-rich structure (see FIG. 1-1). It is considered that since exposure of a charged site of the polyamino acid is increased by the structure change, an electrostatic interaction between the polyamino acid and the protein for medical use in the mixed solution is strengthened, and a formation of a protein for medical use/polyamino acid complex is promoted.

In addition, it is considered that, the alcohol serves to decrease a dielectric constant of the solution, such that an electrostatic interaction between the protein for medical use and the polyamino acid and is strengthened, and cross-linking between protein for medical use/polyamino acid complexes is promoted.

Furthermore, it is considered that since the protein for medical use/polyamino acid complex formed by using alcohol is formed mainly by an electrostatic interaction as a drive force, when a salt is added so as to dissociate the complex, the complex is rapidly dissociated due to an electrostatic shielding effect of the salt (the protein for medical use is released).

This mechanism is based on speculation, and the correctness or incorrectness does not affect the technical scope of the present invention.

As described above, the solution A contains alcohol, that is, alcohol is contained in the mixed solution, such that a particle size (equivalent circle diameter: ECD) of the protein for medical use/polyamino acid complex can be increased. Specifically, in the protein for medical use/polyamino acid complex, a ratio of the number of particles with a particle size of 5 μm or more and 10 μm or less to the number of particles with a particle size of 1 μm or more and less than 5 μm can be increased. Therefore, a preferred embodiment of the present aspect provides a method for producing a protein for medical use/polyamino acid complex in which the solution A contains alcohol, and a ratio of the number of particles of the protein for medical use/polyamino acid complex with a particle size of 5 μm or more and 10 μm or less to the number of particles of the protein for medical use/polyamino acid complex with a particle size of 1 μm or more and less than 5 μm is more than 0.5%. It should be noted that the particle size of the protein for medical use/polyamino acid complex adopts a value measured by a method described in Examples.

### [Hydrophilic Polymer]

Examples of the hydrophilic polymer contained in the solution A in the production method of the present aspect may include, but are not particularly limited to, polyethylene glycol, polypropylene glycol, polyvinyl alcohol, a glycolic acid/L-lactic acid copolymer, polyvinyl pyrrolidone, a hyaluronic acid, and the like. The hydrophilic polymer is preferably selected from the group consisting of polyethylene glycol, polypropylene glycol, polyvinyl alcohol, a glycolic acid/L-lactic acid copolymer, polyvinyl pyrrolidone, and a hyaluronic acid, more preferably selected from polyethylene glycol and polyvinyl pyrrolidone, and still more preferably polyethylene glycol. It should be noted that the hydrophilic polymer herein excludes the polyamino acid and the alcohol described above. In addition, the hydrophilic polymer is referred to as a polymer compound having a water absorption rate of 1% or more when being immersed in a physiological saline solution.

The hydrophilic polymer can be used alone or in a combination of two or more thereof.

In the production method of the present aspect, a mechanism for improving a yield (formation rate) of a protein for medical use/polyamino acid complex by using a hydrophilic polymer is estimated as follows.

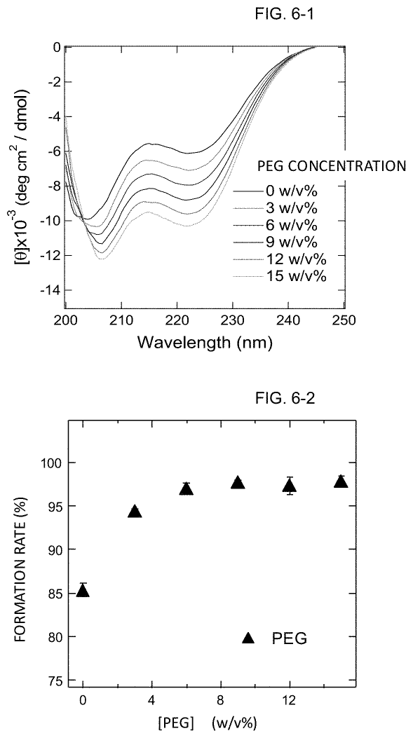

It is considered that the polyamino acid and the hydrophilic polymer are present in the solution A, such that the polyamino acid (for example, a polyglutamic acid) is selectively hydrated by an excluded volume effect of the hydrophilic polymer (for example, polyethylene glycol). That is, it is considered that a surrounding environment of the polyamino acid becomes a polar environment, such that a state in which the charged site of the polyamino acid is exposed to a surface of the polyamino acid becomes stable. Therefore, it is considered that the secondary structure of the polyamino acid is changed to an a-helix-rich structure by exposing the charged site and causing a hydrophobic site to be inside the polyamino acid (see FIG. 6-1). It is considered that since the exposure of a charged site of the polyamino acid is increased by the structure change, an electrostatic interaction between the polyamino acid and the protein for medical use is strengthened, and a formation of a protein for medical use/polyamino acid complex is promoted. In addition, it is considered that, by using a hydrophilic polymer having a large mass, a proportion of a volume occupied by the hydrophilic polymer in the mixed solution is increased, a volume in which the protein for medical use and the polyamino acid can be present is decreased, such that a formation of a protein for medical use/polyamino acid complex is promoted.

In addition, it is considered that when a hydrophilic polymer (for example, an amphiphilic polyethylene glycol) is present in the mixed solution, the protein for medical use, the polyamino acid, and the hydrophilic polymer form a complex by hydrophilic and hydrophobic interactions. Further, it is considered that since the complex and a polyamino acid that does not form a complex are in a mixed state, both an electrostatic interaction and a hydrophobic interaction contribute to a network of these precipitates, and thus it takes time to release the protein for medical use from the complex (dissociation of the complex).

This mechanism is based on speculation, and the correctness or incorrectness does not affect the technical scope of the present invention.

A mass of the hydrophilic polymer can be adequately adjusted depending on a protein for medical use or a polyamino acid to be used. The mass of the hydrophilic polymer is preferably 2000 to 100000 Da, more preferably 2500 to 50000 Da, and still more preferably 3000 to 20000 Da.

### [Solvent]

In the production method of the present aspect, a solvent used for the solution A is not particularly limited, but, for example, a buffer solution that can be generally used in an injection, includes no salt, and can adjust a pH of the mixed solution to more than 4.5 and 0.9 or less, can be used.

Examples of the buffer solution may include a phosphate buffer solution, a citrate buffer solution, a citrate phosphate buffer solution, a histidine buffer solution, a tris-hydrochloric acid buffer solution, an acetate buffer solution, a glycine-NaOH buffer solution, and the like.

### [Method of Preparing Solution A]

In the production method of the present aspect, a method of preparing a solution A is not particularly limited, and an example thereof can include a method of preparing a solution A by (i) preparing a desired polyamino acid in a buffer solution so that the polyamino acid is at a predetermined concentration; (ii) preparing at least one of alcohol or a hydrophilic polymer in a buffer solution so that the alcohol or the hydrophilic polymer is at a predetermined concentration;

and (iii) mixing the solution in (i) with the solution in (ii).

In a preferred embodiment of the present aspect, a method of preparing a solution A by mixing a polyamino acid with at least one of alcohol or a hydrophilic polymer is further included.

It should be noted that concentrations of the polyamino acid, the alcohol, and the hydrophilic polymer in the solution A can be adequately adjusted so that the polyamino acid, the alcohol, and the hydrophilic polymer in a mixed solution are at concentrations as described later.

### <Solution B>

In the production method of the present aspect, a solution B contains a protein for medical use.

### [Protein for Medical Use]

In the production method of the present aspect, the protein for medical use contained in the solution B is not particularly limited, and examples thereof may include an antibody and a fragment thereof, a fusion protein, an enzyme, a hormone, cytokines, and the like.

Examples of the antibody may include muromonab-CD3, trastuzumab, rituximab, palivizumab, infliximab, basiliximab, tocilizumab, gemtuzumab ozogamicin, bepacizumab, ibritumomab tiuxetan, adalimumab, cetuximab, ranibizumab, omalizumab, eculizumab, panitumumab, ustekinumab, golimumab, canakinumab, denosumab, mogamulizumab, certolizumab pegol, ofatumumab, pertuzumab, trastuzumab emtansine, brentuximab vedotin, natalizumab, nipolumab, alemtuzumab, iodine 131-modified tositumomab, catumaxomab, adecatumumab, edrecolomab, abciximab, siltuximab, daclizumab, efalizumab, obinutuzumab, vedolizumab, pembrolizumab, ixekizumab, diridavumab, ipilimumab, belimumab, raxibacumab, ramucirumap, and the like.

Examples of the fusion protein may include etanercept, abatacept, romiplostim, aflibercept, and the like.

Examples of the enzyme may include alteplase, monteplase, imiglucerase, velaglucerase alfa, agalsidase alfa, agalsidase beta, laronidase, alglucosidase alfa, idursulfase, galsulfase, rasburicase, dornase alfa, asparaginase, pegaspargase, condoliase, and the like.

Examples of the hormone may include human insulin, insulin lispro, insulin aspart, insulin glargine, insulin detemir, insulin glulisine, insulin degludec, liraglitide, somatropin, pegvisomant, mecasermin, carperitide, glucagon, follitropin alpha, follitropin beta, teriparatide, metreleptin, and the like.

Examples of the cytokines may include filgrastim, pegfilgrastim, lenograstim, nartograstim, celmoleukin, teceleukin, trafermin, and the like.

In addition to the above protein for medical use, as a protein for medical use, a blood coagulation/fibrinolysis factor, a serum protein, a vaccine, interferons, erythropoietins, and the like can be used.

As a solvent used for a solution B, the same solvent as the solvent used for the solution A can be used.

### [Method of Preparing Solution B]

In the production method of the present aspect, a method of preparing a solution B is not particularly limited, and for example, the solution B can be prepared by mixing a desirable protein for medical use with a buffer solution so that the protein for medical use is at a predetermined concentration. As the buffer solution, the buffer solution used in the preparation method of a solution A can be used.

### <Mixed Solution>

The production method of the present aspect includes obtaining a mixed solution by mixing a solution A with a solution B.

A method of mixing a solution A with a solution B is not particularly limited. The solution B may be added to the solution A, and the solution A may be added to the solution B. Alternatively, the solution A and the solution B may be added to a separate container or the like at the same time.

A content of the polyamino acid in the mixed solution is not particularly limited, but is preferably 0.04 to 2.00 mg/mL. In addition, the content of the polyamino acid in the mixed solution is preferably 0.25 to 1.00 part by mass with respect to 2 parts by mass of the protein for medical use, from the viewpoint of more improving a formation rate of a protein for medical use/polyamino acid complex.

A content of the alcohol in the mixed solution is preferably 2 to 25 mass %, more preferably 3 to 20 mass %, and still more preferably 3 to 15 mass %, with respect to the total amount of the mixed solution, from the viewpoint of more efficiently exerting the effect of the present invention. The dissociation of the protein for medical use when the protein for medical use/polyamino acid complex is redissolved can be adjusted by the content of the alcohol in the mixed solution. When the content of the alcohol is 9 mass % or less, a more rapid release can be expected, and when the content of the alcohol is more than 9 mass %, and preferably 12 mass % or more, a sustained-release effect can be expected.

A content of the hydrophilic polymer in the mixed solution is preferably 2 to 15 mass %, and more preferably 3 to 15 mass %, with respect to the total amount of the mixed solution, from the viewpoint of more efficiently exerting the effect of the present invention. The dissociation of the protein for medical use when the protein for medical use/polyamino acid complex is redissolved can be adjusted by the content of the hydrophilic polymer in the mixed solution. A sustained-release effect can be obtained by increasing the content of the hydrophilic polymer.

A content of the protein for medical use in the mixed solution is not particularly limited, but is preferably 0.5 to 200 mg/mL, more preferably 1 to 100 mg/mL, and still more preferably 1 to 10 mg/mL.

A temperature of the mixed solution, that is, a temperature immediately after the solution A and the solution B are mixed, is not particularly limited, as long as the protein for medical use is not adversely affected, but is preferably 2 to 55° C. and more preferably 4 to 25° C.

A pH of the mixed solution is not particularly limited, but is preferably more than 4.5 and 9.0 or less, which is usable in a general injection. In WO 2015/064591 A, a maximum formation rate of a protein/polyamino acid complex is obtained at a pH away from a pI of the protein by 2.0, and in a case where a protein (for example, human IgG (pI=7.3)) having a pI near a neutral pH is used, it was difficult to obtain a high complex formation rate. On the other hand, in the production method of the present aspect, even when the pH of the mixed solution is close to a pH value of a living body, a high formation rate of a protein for medical use/polyamino complex can be achieved, and the complex can be sufficiently dissociated when being used.

The mixed solution can further contain an additive in addition to the solution A and the solution B, as long as the effect of the present invention is not impaired. The additive contains a substance that is biocompatible and has no adverse effects on a living body even when administered into the living body. Examples of the additive may include sugar, an amino acid, a surfactant such as polysorbate or the like, a preservative such as benzalkonium chloride or the like, and a tonicity agent such as glycerin or the like.

### <Formation of Protein for Medical Use/Polyamino Acid Complex>

The production method of the present aspect includes forming a complex in the mixed solution obtained above.

It is considered that, as described above, the complex is formed in the obtained mixed solution mainly by an electrostatic interaction between the polyamino acid and the protein for medical use. Therefore, a polyamino acid/protein for medical use complex can be formed by allowing the obtained mixed solution to be stand or stirring the obtained mixed solution. The standing or stirring time depends on the amount of mixed solution, the amounts of respective components contained in the mixed solution, and the like. However, the standing or stirring time is, for example, 10 to 30 minutes. In a preferred embodiment, the polyamino acid/protein for medical use complex is formed by allowing the obtained mixed solution to be stand. In addition, a temperature at the time of forming the polyamino acid/protein for medical use complex may be the same as the temperature of the mixed solution.

The formed protein for medical use/polyamino acid complex can be collected, for example, by centrifugation. The collected protein for medical use/polyamino acid complex can be washed, if necessary.

For example, in a case where the solution A contains alcohol, the alcohol can be removed by washing the collected protein for medical use/polyamino acid complex. The washing can be performed by a known method in the related art. As described later, since the protein for medical use/polyamino acid complex can be dissociated by using an electrolyte, a solution containing no electrolyte is used in the washing. As the solution containing no electrolyte, the above buffer solution can be used.

It is considered that since the protein for medical use/polyamino acid complex obtained by the production method of the present aspect is formed by a weak electrostatic interaction, the protein for medical use/polyamino acid complex is an irreversible complex that is not dissociated even when stirred with a buffer solution. Therefore, a yield of the protein for medical use obtained by dissociating the complex can be maintained even when the protein for medical use/polyamino acid complex is washed.

### <Dissociation (Redissolution) of Protein for Medical Use/Polyamino Acid Complex>

The protein for medical use can be obtained by dissociating (redissolving) the protein for medical use/polyamino acid complex according to the present aspect by using an electrolyte. Therefore, the protein for medical use/polyamino acid complex can be used for the following uses.

As a method of redissolving a protein for medical use/polyamino acid complex, the protein for medical use/polyamino acid complex can be dissociated by adding an electrolyte to a solution (for example, the mixed solution) containing the complex.

Examples of the electrolyte can include NaCl, KCl, CaCl₂, MgCl₂, and the like. The electrolyte is preferably NaCl from the viewpoint of biocompatibility.

A concentration of the electrolyte is not particularly limited, but is preferably 150 to 300 mM.

### <Use of Protein for Medical Use/Polyamino Acid Complex>

As the protein for medical use/polyamino acid complex according to the present aspect, the mixed solution containing the protein for medical use/polyamino acid complex can be used as it is. In addition, the protein for medical use/polyamino acid complex collected from the mixed solution can also be used. In particular, in the production method of the present aspect, since the yield of the protein for medical use/polyamino acid complex can be stably improved, the protein for medical use/polyamino acid complex is precipitated by centrifugation or the like, and a solution containing an electrolyte is used, thereby obtaining and using a solution containing a highly concentrated protein for medical use/polyamino acid complex.

The protein for medical use/polyamino acid complex and the solution containing the complex according to the present aspect can be used as a protein drug for any route of an administration, such as an oral, hypodermic, intraperitoneal, pulmonary, or intranasal administration, and can be used for a topical treatment of an intralesional administration, if necessary. In addition, the protein for medical use/polyamino acid complex according to the present aspect may be formulated as a form of an injection, or a form of a drug for a catheter disposed close to a desired position. In addition, an excipient or a diluting agent that is pharmaceutically acceptable may be contained depending on an administration form or an administration route.

### <<Protein for Medical Use/Polyamino Acid Complex>>

An aspect of the present invention is a complex of a protein for medical use and a polyamino acid. By forming the complex of the protein for medical use and the polyamino acid, it is possible to implement a simple concentration and a stress resistance of the protein for medical use.

Since the descriptions of “protein for medical use”, “polyamino acid”, “hydrophilic polymer”, and “use of protein for medical use/polyamino acid complex” in the present aspect are as in the production method of a protein for medical use/polyamino acid complex, the description thereof are omitted.

A preferred embodiment of the present aspect is a complex of a protein for medical use, a polyamino acid, and a hydrophilic polymer. By forming the complex of the protein for medical use, the polyamino acid, and the hydrophilic polymer, a sustained-release time of the protein for medical use at the time of administration to a patient and the like can be controlled by adjusting the amount of hydrophilic polymer contained in the complex. The amount of hydrophilic polymer contained in the complex can be adjusted by a content of the hydrophilic polymer contained in the complex in the mixed solution.

In the present aspect, the polyamino acid is preferably selected from the group consisting of a polyglutamic acid, polylysine, polyarginine, and water-soluble salts thereof, from the viewpoint that such polyamino acid has biocompatibility, and a pI at which the polyamino acid easily forms a complex with a protein for medical use, and the like.

In the present aspect, the hydrophilic polymer is preferably selected from the group consisting of polyethylene glycol, polypropylene glycol, polyvinyl alcohol, a glycolic acid/L-lactic acid copolymer, polyvinyl pyrrolidone, and a hyaluronic acid.

### EXAMPLES

Hereinafter, the present invention will be described by using specific examples, but the present invention is not limited by these examples.

### Test Example 1

### Example 1-1

### (Preparation of Solution e1 Containing Polyamino Acid)

In a 10 mM citrate buffer solution (pH 5.0), a poly-L-glutamic acid (polyE, mass: 3 kDa to 15 kDa) was prepared so that a concentration of the poly-L-glutamic acid was 1.5 mg/mL, thereby preparing a solution e1.

### (Preparation of Solution a1 Containing Alcohol)

In a 10 mM citrate buffer solution (pH 5.0), ethanol was prepared so that a concentration of the ethanol was 9% (v/v), thereby preparing a solution a1.

### (Preparation of Solution A1)

50 μL of each of the solution e1 and the solution a1 were mixed, thereby preparing 100 μL of a solution A1.

(Measurement of Far Ultraviolet CD Spectrum of polyE)

Separately, 50 μL of each of the solution e1, the solution a1, and a 10 mM citrate buffer solution (pH 5.0) were mixed, thereby preparing 150 μL of a solution A1. A far ultraviolet CD spectrum (measuring apparatus: JASCO J-720, manufactured by JASCO Corporation) of polyE in the solution A1 was measured. The results are shown in FIG. 1-1.

### (Preparation of Solution B)

In a 10 mM citrate buffer solution (pH 5.0), human immunoglobulin (IgG) was prepared so that a concentration of the human immunoglobulin was 6.0 mg/mL, thereby preparing a solution B.

### (Formation of Complex)

50 μL of the solution B was mixed with 100 μL of the solution A1 prepared above, thereby preparing a mixed solution. The mixed solution was allowed to stand at 25° C. for 30 minutes, thereby forming a human immunoglobulin/poly-L-glutamic acid (IgG/polyE) complex.

### (Calculation of Formation Rate of Complex)

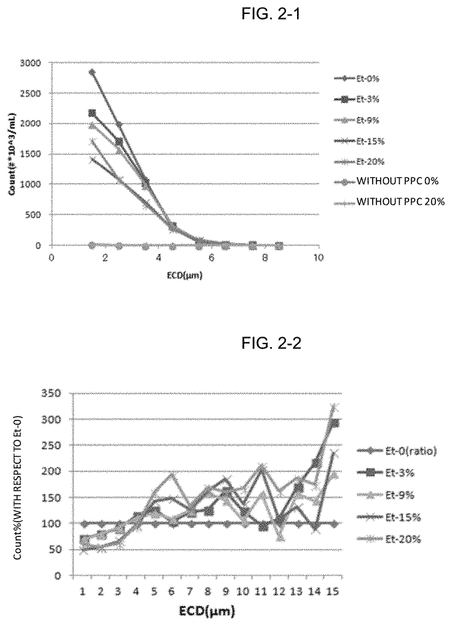

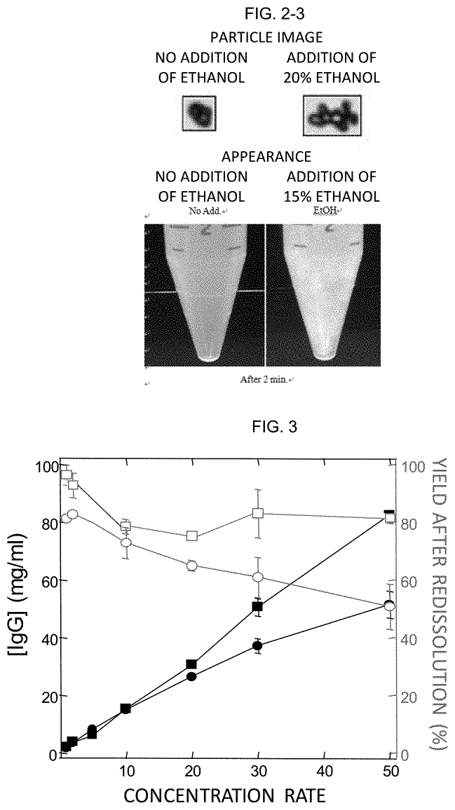

After the IgG/polyE complex was formed, centrifugation was performed at 9000×g for 5 minutes, thereby precipitating the IgG/polyE complex. A supernatant was collected, and an IgG concentration in the supernatant was measured by UV measuring instrument (ND-1000, manufactured by LMS Co., Ltd.), thereby calculating a formation rate of the IgG/polyE complex. For example, when the IgG concentration in the supernatant is 0%, the entire IgG forms the complex, thus a formation rate of the IgG/polyE complex is 100%. The results are shown in FIG. 1-2.

### Examples 1-2 to 1-15

An IgG/polyE complex was formed and a formation rate thereof was calculated in the same manner as that of Example 1-1, except for the following changes:
in the preparation of the solution a1 containing alcohol, ethanol, methanol, or trifluoroethanol (TFE) was prepared so that a concentration thereof was set as shown in Table 1, thereby preparing solutions a2 to a15;
in the preparation of the solution A1, each of the solutions a2 to a15 was used instead of the solution a1, thereby preparing solutions A2 to A15. In addition, a far ultraviolet CD spectrum of polyE in each of the solutions A2 to A5 was measured in the same manner as that of Example 1-1. The results are shown in FIG. 1-1; and
in the formation of an IgG/polyE complex, each of the solutions A2 to A15 was used instead of the solution A1, thereby preparing a mixed solution.

The results obtained by calculating the formation rates of the complexes are shown in FIG. 1-2.

TABLE 1

| | Ethanol | Methanol | TFE |
|---|---|---|---|
| Solution No. | % (v/v) | % (v/v) | % (v/v) |
| a1 | 9 | — | — |
| a2 | 18 | — | — |
| a3 | 27 | — | — |
| a4 | 36 | — | — |
| a5 | 45 | — | — |
| a6 | — | 9 | — |
| a7 | — | 18 | — |
| a8 | — | 27 | — |
| a9 | — | 36 | — |
| a10 | — | 45 | — |
| a11 | — | — | 9 |
| a12 | — | — | 18 |
| a13 | — | — | 27 |
| a14 | — | — | 36 |
| a15 | — | — | 45 |

As a buffer, a 10 mM citrate buffer solution (pH 5.0) was used

In the preparation of the solution A1, an IgG/polyE complex was formed, and a formation rate thereof was calculated, in the same manner as that of Example 1-1, except that a 10 mM citrate buffer solution (pH 5.0) was used instead of the solution a1 to prepare a solution A′1. The results are shown in FIG. 1-2.

In addition, a far ultraviolet CD spectrum of polyE in the solution A′1 was measured in the same manner as that of Example 1-1. The results are shown in FIG. 1-1.

The mixed solutions prepared in Test Example 1 are shown in Table 2.

TABLE 2

| | Mixed solution | | | | | | | | |
|---|---|---|---|---|---|---|---|---|---|
| | | | | | | | | Solution B | |
| | Solution A | | | | | | | Protein for | |
| | | Polyamino acid | | Alcohol | | | | medical use | |
| | | Solution | polyE | Solution | Ethanol % | Methanol % | TFE % | Human IgG | |
| | No. | No. | (mg/mL) | No. | (v/v) | (v/v) | (v/v) | (mg/mL) | pH |
| Example 1-1 | A1 | e1 | 0.5 | a1 | 3 | — | — | 2.0 | 5.0 |
| Example 1-2 | A2 | e1 | 0.5 | a2 | 6 | — | — | 2.0 | 5.0 |
| Example 1-3 | A3 | e1 | 0.5 | a3 | 9 | — | — | 2.0 | 5.0 |
| Example 1-4 | A4 | e1 | 0.5 | a4 | 12 | — | — | 2.0 | 5.0 |
| Example 1-5 | A5 | e1 | 0.5 | a5 | 15 | — | — | 2.0 | 5.0 |
| Example 1-6 | A6 | e1 | 0.5 | a6 | — | 3 | — | 2.0 | 5.0 |
| Example 1-7 | A7 | e1 | 0.5 | a7 | — | 6 | — | 2.0 | 5.0 |
| Example 1-8 | A8 | e1 | 0.5 | a8 | — | 9 | — | 2.0 | 5.0 |
| Example 1-9 | A9 | e1 | 0.5 | a9 | — | 12 | — | 2.0 | 5.0 |
| Example 1-10 | A10 | e1 | 0.5 | a10 | — | 15 | — | 2.0 | 5.0 |
| Example 1-11 | A11 | e1 | 0.5 | a11 | — | — | 3 | 2.0 | 5.0 |
| Example 1-12 | A12 | e1 | 0.5 | a12 | — | — | 6 | 2.0 | 5.0 |
| Example 1-13 | A13 | e1 | 0.5 | a13 | — | — | 9 | 2.0 | 5.0 |
| Example 1-14 | A14 | e1 | 0.5 | a14 | — | — | 12 | 2.0 | 5.0 |
| Example 1-15 | A15 | e1 | 0.5 | a15 | — | — | 15 | 2.0 | 5.0 |
| Comparative | A′ 1 | e1 | 0.5 | — | — | — | — | 2.0 | 5.0 |
| Example 1 | | | | | | | | | |

polyE: polyglutamic acid (3 kDa to 15 kDa)
TFE: trifluoroethanol

### (Discussion)

As shown in FIG. 1-1, it can be appreciated that as a content of the alcohol in the mixed solution is increased, a polyglutamic acid forms an a-helix-rich structure.

In addition, as shown in FIG. 1-2, it can be appreciated that the solution A contains alcohol, that is, alcohol is present in the mixed solution, such that a formation rate of an IgG/polyE complex can be improved.

### Test Example 2

### Examples 2-1 to 2-4

### (Preparation of Solution a16 Containing Alcohol)

In a 10 mM citrate buffer solution (pH 5.0), ethanol was prepared so that a concentration of the ethanol was 60% (v/v), thereby preparing a solution a16.

### (Preparation of Solution A16)

50 μL of each of a solution e1 and a solution a16 were mixed, thereby preparing 100 μL of a solution A1.

### (Formation of Complex)

50 μL of each of the solutions B was mixed with 100 μL of each of the solutions A1, A3, A5, and A16 prepared above, thereby preparing a mixed solution. Each mixed solution was allowed to stand at 25° C. for 30 minutes, thereby forming an IgG/polyE complex.

### (Particle Size and Particle Shape)

After the IgG/polyE complex was formed, the IgG/polyE complex was diluted about 300-fold with a 10 mM citrate buffer solution (pH 5.0), and a distribution of particle sizes (equivalent circle diameter: ECD) and particle shape data were obtained by using MicroFlow Imaging (manufactured by Brightwell). The results are shown in FIGS. 2-1 and 2-2.

### Comparative Example 2-1

50 μL of the solution B was mixed with 100 μL of the A′1 prepared above, thereby preparing a mixed solution. The mixed solution was allowed to stand at 25° C. for 30 minutes, thereby forming an IgG/polyE complex. Thereafter, the IgG/polyE complex was diluted about 300-fold with a 10 mM citrate buffer solution (pH 5.0), and a distribution of particle sizes and particle shape data were obtained by using MicroFlow Imaging (manufactured by Brightwell). The results are shown in FIGS. 2-1 and 2-2.

### Comparative Example 2-2

In a 10 mM citrate buffer solution (pH 5.0), ethanol was prepared so that a concentration of the ethanol was 60% (v/v), thereby preparing a solution a16. 50 μL of the solution a16 was mixed with 50 μL of a 10 mM citrate buffer solution (pH 5.0), thereby preparing 100 μL of a solution A′2. 50 μL of a solution B was mixed with 100 μL of the solution A′2, thereby preparing a mixed solution. The mixed solution was allowed to stand at 25° C. for 30 minutes. Thereafter, the IgG/polyE complex was diluted at a concentration suitable for measurement with a 10 mM citrate buffer solution (pH 5.0), and a distribution of particle sizes (equivalent circle diameter: ECD) and particle shape data were obtained by using MicroFlow Imaging (manufactured by Brightwell). The results are shown in FIGS. 2-1 and 2-2.

### Comparative Example 2-3

100 μL of a 10 mM citrate buffer solution (pH 5.0) was mixed with 50 μL of a solution B, and the mixed solution was allowed to stand at 25° C. for 30 minutes. Thereafter, the IgG/polyE complex was diluted about 300-fold with a 10 mM citrate buffer solution (pH 5.0), and a distribution of particle sizes and particle shape data were obtained by using MicroFlow Imaging (manufactured by Brightwell). The results are shown in FIGS. 2-1 and 2-2.

The mixed solutions prepared in Test Example 2 are shown in Table 3.

TABLE 3

| | Mixed solution | | | | | | |
|---|---|---|---|---|---|---|---|
| | | | | | | Solution B | |
| | Solution A | | | | | Protein for | |
| | | Polyamino acid | | Alcohol | | medical use | |
| | | | polyE | | Ethanol % | Human IgG | |
| | No. | No. | (mg/mL) | No. | (v/v) | (mg/mL) | pH |
| Example 2-1 | A1 | e1 | 0.5 | a1 | 3 | 2.0 | 5.0 |
| Example 2-2 | A3 | e1 | 0.5 | a3 | 9 | 2.0 | 5.0 |
| Example 2-3 | A5 | e1 | 0.5 | a4 | 12 | 2.0 | 5.0 |
| Example 2-4 | A16 | e1 | 0.5 | a16 | 20 | 2.0 | 5.0 |
| Comparative | A′ 1 | e1 | 0.5 | — | — | 2.0 | 5.0 |
| Example 2-1 | | | | | | | |
| Comparative | A′ 2 | — | — | a16 | 20 | 2.0 | 5.0 |
| Example 2-2 | | | | | | | |
| Comparative | —- | — | — | — | — | 2.0 | 5.0 |
| Example 2-3 | | | | | | | |

polyE: polyglutamic acid (3 kDa to 15 kDa)

### (Discussion)

As shown in FIG. 2-1, it can be appreciated that in Comparative Examples 2-2 and 2-3, a complex was not formed, and thus particles were hardly observed.

In addition, as shown in FIG. 2-1, it can be appreciated that in each of Examples 2-1 to 2-4 and Comparative Example 2-1, an IgG/polyE complex was formed, and ECD thereof was about 1 to 5 μm in many particles.

As shown in FIG. 2-2, it can be appreciated that in Examples 2-1 to 2-4, a mixed solution contains alcohol, such that a proportion of particles having ECD of 6 μm or more was increased, unlike Comparative Example 2-1.

A ratio of the number of particles having ECD of 5 μm or more and 10 μm or less to the number of particles having ECD of 1 μm or more and less than 5 μm is shown in Table 4.

TABLE 4

| | Ethanol concentration in mixed solution | | | | |
|---|---|---|---|---|---|
| | 0% (v/v) | 3% (v/v) | 9% (v/v) | 15% (v/v) | 20% (v/v) |
| Ratio of | 0.440 | 0.591 | 0.655 | 1.121 | 1.269 |
| number of | | | | | |
| particles | | | | | |
| (%) | | | | | |

As shown in Table 4, it can be appreciated that the ratio of the number of particles having ECD of 5 μm or more and 10 μm or less to the number of particles having ECD of 1 μm or more and less than 5 μm was more than 0.5% by adding alcohol. In addition, it can be appreciated that a proportion of particles having a larger structure can be increased by raising a concentration of the alcohol.

### Test Example 3

### Example 3

300 μL of each of a solution e1 and a solution a5 were mixed, thereby preparing 600 μL of a solution A5. 300 μL of a solution B was mixed with 600 μL of the solution A5, thereby preparing a mixed solution. The mixed solution was allowed to stand at 25° C. for 30 minutes, thereby forming an IgG/polyE complex. After the IgG/polyE complex was formed, centrifugation was performed at 9000×g for 5 minutes, thereby precipitating the IgG/polyE complex. 885 μL of a supernatant was collected, an IgG concentration in the supernatant was measured with a UV spectrum of 280 nm, and a formation rate of an IgG/polyE complex was obtained, thereby calculating the amount of IgG precipitated as the IgG/polyE complex.

To a solution containing the IgG/polyE complex from which the supernatant was removed, a 150 to 900 mM NaCl-10 mM citrate buffer solution (pH 7.0) was added so that a NaCl concentration was 150 mM, and then the solution was allowed to stand at 25° C. for 1 hour, thereby redissolving the IgG/polyE complex. Centrifugation was performed at 9000×g for 5 minutes, thereby precipitating an undissociated IgG/polyE complex. The supernatant was collected, and the IgG concentration in the supernatant was measured with a UV spectrum of 280 nm, and then a yield of IgG was obtained, thereby calculating the amount of IgG collected at the time of the redissolution.

It should be noted that a NaCl concentration in a living body is 150 mM, and the present Example imitates the release at the time of injecting the IgG/polyE complex into the living body.

### Comparative Example 3

300 μL of a solution B was mixed with 600 μL of a solution A′1, thereby preparing a mixed solution. The mixed solution was allowed to stand at 25° C. for 30 minutes, thereby forming an IgG/polyE complex. After the IgG/polyE complex was formed, centrifugation was performed at 9000×g for 5 minutes, thereby precipitating the IgG/polyE complex. 885 μL of a supernatant was collected, an IgG concentration in the supernatant was measured with a UV spectrum of 280 nm, and a formation rate of an IgG/polyE complex was obtained, thereby calculating the amount of IgG precipitated as the IgG/polyE complex.

To a solution containing the IgG/polyE complex from which the supernatant was removed, a 150 to 900 mM NaCl-10 mM citrate buffer solution (pH 7.0) was added so that a NaCl concentration was 150 mM, and then the solution was allowed to stand at 25° C. for 1 hour, thereby redissolving (dissociating) the IgG/polyE complex. Centrifugation was performed at 9000×g for 5 minutes, thereby precipitating an undissociated IgG/polyE complex. The supernatant was collected, and the IgG concentration in the supernatant was measured with a UV spectrum of 280 nm, and then a yield of IgG was obtained, thereby calculating the amount of IgG collected at the time of the redissolution.

### (Yield After Redissolution)

A recovery rate of IgG after the redissolution was calculated by using the following Equation (1).

$$[ Equation 1 ]$$

$$IgG recovery rate ( \% ) after redissolution = \frac{\begin{matrix}Amount of IgG collected \\ at the time of redissolution\end{matrix}}{Amount of IgG precipitated as complex} \times 100 \qquad Equation ( 1 )$$

The results are shown in FIG. 3. It should be noted that, in FIG. 3, a concentration rate was calculated by the following equation.

Concentration rate=(Liquid amount after redissolution)/(Liquid amount at the time of precipitation) [Equation 2]

### (Discussion)

As shown in FIG. 3, it can be appreciated that, in Example 3, since ethanol was contained in the mixed solution, the IgG/polyE complex was dissociated by 95% or more by being allowed to be stand for 1 hour.

In addition, it can be appreciated that, in Example 3, 80% of IgG can be collected even at the time of 50-fold concentration (about 100 mg/mL). A recovery rate of IgG in Example 3 was 1.6 times that of IgG in Comparative Example 3.

### Test Example 4

### Examples 4-1 to 4-5

300 μL of a solution e1 was mixed with 300 μL of each of solutions a1 to a5, thereby preparing 600 μL of solutions A1 to A5. 300 μL of a solution B was mixed with 600 μL of each of the solutions A1 to A5, thereby preparing a mixed solution. The mixed solution was allowed to stand at 25° C. for 30 minutes, thereby forming an IgG/polyE complex. After the IgG/polyE complex was formed, centrifugation was performed at 9000×g for 5 minutes, and the IgG/polyE complex was precipitated and 885 μL of a supernatant was removed.

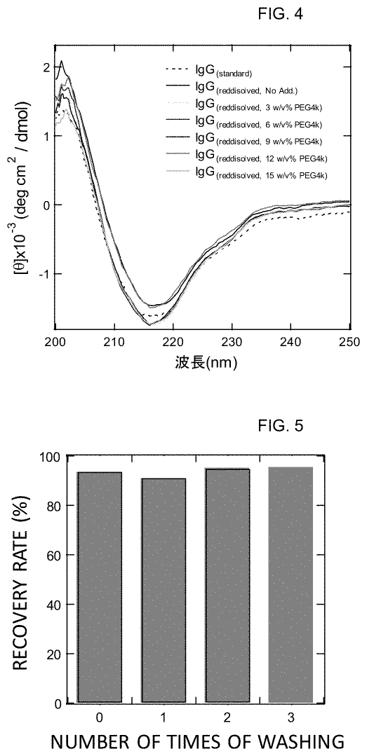

To a solution containing the IgG/polyE complex from which the supernatant was removed, 3 to 885 μL of a 150 to 900 mM NaCl-10 mM citrate buffer solution (pH 7.0) was added, and then the solution was allowed to stand at 25° C. for 1 hour, thereby redissolving the IgG/polyE complex. Centrifugation was performed at 9000×g for 5 minutes, thereby precipitating an undissociated IgG/polyE complex. The supernatant was collected, and then a concentration of IgG in the supernatant was measured with a UV spectrum of 280 nm. The concentration of IgG contained in the supernatant was adjusted to 0.1 mg/mL using a 150 mM NaCl-10 mM citrate buffer solution (pH 7.0). A far ultraviolet CD spectrum (measuring apparatus: JASCO J-720, manufactured by JASCO Corporation) of IgG in a wavelength of 200 to 250 nm was measured in the adjusted supernatant. The cumulative number of times of measurements was 15. The results are shown in FIG. 4.

In FIG. 4, a far ultraviolet CD spectrum (measuring apparatus: JASCO J-720, manufactured by JASCO Corporation) of IgG in a wavelength of 200 to 250 nm was measured in the solution B, and was used as a standard.

### Reference Example 4

A far ultraviolet CD spectrum (measuring apparatus: JASCO J-720, manufactured by JASCO Corporation) of IgG in a wavelength of 200 to 250 nm was measured in the same manner as those of Examples 4-1 to 4-5, except that a solution A′1 was used instead of the solutions A1 to A5. The results are shown in FIG. 4.

### (Discussion)

As shown FIG. 4, it can be appreciated that a change in structure of IgG was not observed even after the formation and dissociation of the IgG/polyE complex were performed by using ethanol.

### Test Example 5

50 μL of each of a solution e1 and a solution a5 were mixed, thereby preparing 100 μL of a solution A5. 50 μL of a solution B was mixed with 100 μL of the solution A5, thereby preparing a mixed solution. The mixed solution was allowed to stand at 25° C. for 30 minutes, thereby forming an IgG/polyE complex. After the IgG/polyE complex was formed, centrifugation was performed at 9000×g for 5 minutes, thereby precipitating the IgG/polyE complex.

The obtained IgG/polyE complex was washed with a 10 mM citrate buffer solution (pH 5.0) 0 to 3 times (when the washing was performed 0 times, (ii) the following procedure was not performed). A washing operation was performed according to the following procedures.

(i) 135 μL of a supernatant was removed, and then 135 μL of a 10 mM citrate buffer solution (pH 5.0) was added;

(ii) pipetting was performed 15 to 30 times;

(iii) centrifugation was performed at 9000×g for 5 minutes; and

(iv) 135 μL of a supernatant was removed.

After operations (i) to (iv) were repeated 1 to 3 times, the IgG/polyE complex was redissolved with a 150 mM NaCl-10 mM citrate buffer solution (pH 7.0), and an IgG concentration was measured with a UV spectrum of 280 nm, thereby obtaining a yield of IgG. It should be noted that an ethanol concentration is theoretically decreased up to 0.015% (=15%×10⁻³) by performing washing 3 times. The results are shown in FIG. 5.

### (Discussion)

As shown in FIG. 5, the washing operation after the precipitation of the IgG/polyE complex did not affect the yield of IgG. Accordingly, it can be appreciated that alcohol can be removed by repeating the washing operation.

### Test Example 6

### Examples 6-1 to 6-5

### (Preparation of Solutions p1 to p5 Containing Polyethylene Glycol)

In a 10 mM citrate buffer solution (pH 5.0), polyethylene glycol (PEG) (4000 Da) was prepared so that a concentration of the PEG was set as shown in Table 5, thereby preparing solutions p1 to p5.

TABLE 5

| | PEG (4 kDa) | PEG (20 kDa) | |
|---|---|---|---|
| No. | % (w/v) | % (w/v) | Buffer solution |
| p1 | 9 | — | 10 mM citrate buffer |
| | | | solution (pH 5.0) |
| p2 | 18 | — | 10 mM citrate buffer |
| | | | solution (pH 5.0) |
| p3 | 27 | — | 10 mM citrate buffer |
| | | | solution (pH 5.0) |
| p4 | 36 | — | 10 mM citrate buffer |
| | | | solution (pH 5.0) |
| p5 | 45 | — | 10 mM citrate buffer |
| | | | solution (pH 5.0) |

PEG: polyethylene glycol

### (Preparation of Solutions A17 to A21)

50 μL of a solution e1 and 50 μL of each of the solutions p1 to p5 were mixed, thereby preparing 100 μL of solutions A16 to A20.

(Measurement of Far Ultraviolet CD Spectrum of polyE)

Separately, 50 μL of the solution e1, 50 μL of each of the solutions p1 to p5, and 50 μL of a 10 mM citrate buffer solution (pH 5.0) were mixed, thereby preparing 150 μL of solutions A16 to A20. A far ultraviolet CD spectrum (measuring apparatus: JASCO J-720, manufactured by JASCO Corporation) of polyE in each of the solutions A17 to A21 was measured. The results are shown in FIG. 6-1.

### (Formation of Complex)

50 μL of a solution B was mixed with 100 μL of each of the solutions A17 to A21, thereby preparing a mixed solution. Each mixed solution was allowed to stand at 25° C. for 30 minutes, thereby forming an IgG/polyE/PEG complex.

### (Calculation of Formation Rate of Complex)

After the IgG/polyE/PEG complex was formed, centrifugation was performed at 9000×g for 5 minutes, thereby precipitating the complex. A supernatant was collected, and an IgG concentration in the supernatant was measured with ND-1000 (manufactured by LMS Co., Ltd.), thereby calculating a formation rate of the complex. The results are shown in FIG. 6-2. It should be noted that the formation rate of the complex was calculated by the following equation.

Formation rate of complex=((IgG concentration after redissolution)/(Initial IgG concentration−Supernatant concentration after precipitation))×100 [Equation 3]

### Comparative Example 6

A formation rate of a complex was calculated in the same manner as those of Examples 6-1 to 6-5, except that a solution A′1 was used instead of the solutions A17 to A21. The results are shown in FIG. 6-2.

In addition, a far ultraviolet CD spectrum of polyE in the solution A′1 was measured. The results are shown in FIG. 6-1.

The mixed solutions prepared in Test Example 6 are shown in Table 6.

TABLE 6

| | Mixed Soultion | | | | | | |
|---|---|---|---|---|---|---|---|
| | | | | | | Solution B | |
| | Solution A | | | | | Protein for | |
| | | Polyamino acid | | PEG | | medical use | |
| | | | polyE | | % | Human IgG | |
| | No. | No. | (mg/mL) | No. | (w/v) | (mg/mL) | pH |
| Example 6-1 | A17 | e1 | 0.5 | p1 | 3 | 2.0 | 5.0 |
| Example 6-2 | A18 | e1 | 0.5 | p2 | 6 | 2.0 | 5.0 |
| Example 6-3 | A19 | e1 | 0.5 | p3 | 9 | 2.0 | 5.0 |
| Example 6-4 | A20 | e1 | 0.5 | p4 | 12 | 2.0 | 5.0 |
| Example 6-5 | A21 | e1 | 0.5 | p5 | 15 | 2.0 | 5.0 |
| Comparative | A′ 1 | e1 | 0.5 | — | — | 2.0 | 5.0 |
| Example 6 | | | | | | | |

polyE: polyglutamic acid (3 kDa to 15 kDa)
PEG: polyethylene glycol (4000 Da)

### (Discussion)

As shown in FIG. 6-1, it can be appreciated that as a PEG concentration in the mixed solution is increased, a polyglutamic acid forms an a-helix-rich structure.

In addition, as shown in FIG. 6-2, it can be appreciated that a formation rate of the IgG/polyE/PEG complex can be improved by having the mixed solution include PEG.

### Test Example 7

### Examples 7-1 to 7-5

50 μL of a solution e1 and 50 μL of each of solutions p1 to p5 were mixed, thereby preparing 100 μL of solutions A17 to A21. 50 μL of a solution B was mixed with 100 μL of each of the solutions A17 to A21, thereby preparing a mixed solution. Each mixed solution was allowed to stand at 25° C. for 30 minutes, thereby forming an IgG/polyE/PEG complex. After the IgG/polyE/PEG complex was formed, centrifugation was performed at 9000×g for 5 minutes, and the IgG/polyE/PEG complex was precipitated and 135 μL of a supernatant was removed.

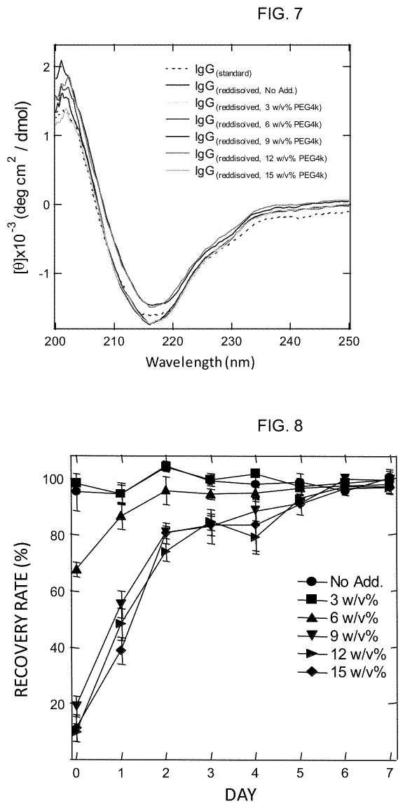

To a solution containing the IgG/polyE/PEG complex from which the supernatant was removed, 135 μL of a 150 mM NaCl-10 mM citrate buffer solution (pH 7.0) was added, and then the solution was allowed to stand at 25° C. for 1 hour, thereby redissolving the IgG/polyE/PEG complex. Centrifugation was performed at 9000×g for 5 minutes, thereby precipitating an undissociated IgG/polyE complex. The supernatant was collected, and then a concentration of IgG in the supernatant was measured with a UV spectrum of 280 nm. The concentration of IgG contained in the supernatant was adjusted to 0.1 mg/mL using a 150 mM NaCl-10 mM citrate buffer solution (pH 7.0). A far ultraviolet CD spectrum (measuring apparatus: JASCO J-720, manufactured by JASCO Corporation) of IgG in a wavelength of 200 to 250 nm was measured in the adjusted supernatant. The cumulative number of times of measurements was 15. The results are shown in FIG. 7.

In FIG. 7, a far ultraviolet CD spectrum (measuring apparatus: JASCO J-720, manufactured by JASCO Corporation) of IgG in a wavelength of 200 to 250 nm was measured in the solution B, and was used as a standard.

### Reference Example 7

A far ultraviolet CD spectrum (measuring apparatus: JASCO J-720, manufactured by JASCO Corporation) of IgG in a wavelength of 200 to 250 nm was measured in the same manner as those of Examples 7-1 to 7-5, except that a solution A′1 was used instead of the solutions A17 to A21. The results are shown in FIG. 7.

### (Discussion)

As shown in FIG. 7, it can be appreciated that a structure of IgG was not changed even after an IgG/polyE/PEG complex was formed by adding PEG, and the complex was dissociated.

### Test Example 8

### Examples 8-1 to 8-5

50 μL of a solution e1 and 50 μL of each of solutions p1 to p5 were mixed, thereby preparing 100 μL of solutions A17 to A21. 50 μL of a solution B was mixed with 100 μL of each of the solutions A17 to A21, thereby preparing a mixed solution. Each mixed solution was allowed to stand at 25° C. for 30 minutes, thereby forming an IgG/polyE/PEG complex. After the IgG/polyE/PEG complex was formed, centrifugation was performed at 9000×g for 5 minutes, and the IgG/polyE/PEG complex was precipitated and 135 μL of a supernatant was removed.

To a solution containing the IgG/polyE/PEG complex from which the supernatant was removed, 135 μL of a 150 mM NaCl-10 mM citrate buffer solution (pH 7.0) was added, and then the solution was allowed to stand at 25° C. for 1 hour (0 days) and for 1 to 5 days, thereby redissolving (dissociating) the IgG/polyE/PEG complex.

Centrifugation was performed at 9000×g for 5 minutes, thereby precipitating an undissociated complex. The supernatant was collected, and then an IgG concentration in the supernatant was measured with a UV spectrum of 280 nm, thereby calculating a recovery rate of IgG. The results are shown in FIG. 8.

### Reference Example 8

A recovery rate of IgG was calculated by measuring an IgG concentration in the same manner as those of Examples 8-1 to 8-5, except that a solution A′1 was used instead of the solutions A17 to A21. The results are shown in FIG. 8.

### (Discussion)

As shown in FIG. 8, when a PEG concentration contained in the mixed solution was 3 or 6%, 95% of IgG was rapidly released (recovery rate: 95%). In addition, when the PEG concentration contained in the mixed solution was 9 to 15%, a sustained-release effect was observed. Almost 100% of a recovery rate of IgG contained in the complex could be maintained up to 4 days after the redissolution was initiated, in any sample of Examples 8-1 to 8-5.

### Test Example 9

### Examples 9-1 to 9-3

### (Preparation of Solution e2 Containing Polyamino Acid)

In a 10 mM citrate buffer solution (pH 6.0), a poly-L-glutamic acid (polyE, mass: 50 kDa to 100 kDa) was prepared so that a concentration of the poly-L-glutamic acid was 1.5 mg/mL, thereby preparing a solution e2.

### (Preparation of Solutions p6 to p8 Containing Polyethylene Glycol)

In a 10 mM citrate buffer solution (pH 6.0), polyethylene glycol (PEG) (4000 Da) was prepared so that the PEG was at a predetermined concentration in the mixed solution shown in Table 7, thereby preparing solutions p6 to p8.

### (Preparation of Solutions A22 to A24)

50 μL of a solution e2 and 50 μL of each of solutions p6 to p8 were mixed, thereby preparing 100 μL of solutions A22 to A24.

### (Formation of Complex)

50 μL of a solution B was mixed with 100 μL of each of the solutions A22 to A24, thereby preparing a mixed solution. Each mixed solution was allowed to stand at 25° C. for 30 minutes, thereby forming an IgG/polyE/PEG complex.

### (Calculation of Formation Rate of Complex)

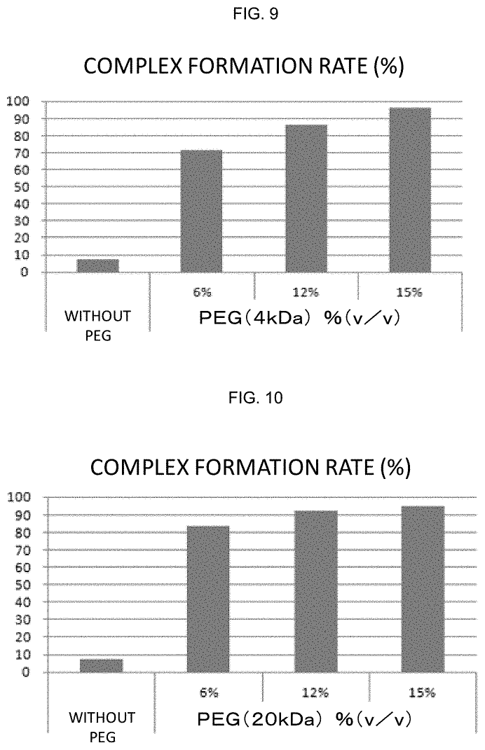

After the IgG/polyE/PEG complex was formed, centrifugation was performed at 9000×g for 5 minutes, thereby precipitating the IgG/polyE/PEG complex. A supernatant was collected, and an IgG concentration in the supernatant was measured by size exclusion chromatography (SEC) (used column: TSK-GEL G3000SWXL, particle size: 5 μm, manufacture by TOSOH Corporation, measuring apparatus: high performance liquid chromatography LC20A, manufactured by Shimadzu Corporation), thereby calculating a formation rate of the IgG/polyE/PEG complex. The results are shown in FIG. 9 and Table 7.

### Comparative Example 9

### (Preparation of Solution A′3)

50 μL of a solution e2 was mixed with 50 μL of a 10 mM citrate buffer solution (pH 6.0), thereby preparing 100 μL of a solution A′3.

### (Formation of Complex)

50 μL of a solution B was mixed with 100 μL of a solution A′3, thereby preparing a mixed solution. Each mixed solution was allowed to stand at 25° C. for 30 minutes, thereby forming an IgG/polyE complex.

### (Calculation of Formation Rate of Complex)

After the IgG/polyE complex was formed, centrifugation was performed at 9000×g for 5 minutes, thereby precipitating the IgG/polyE complex. A supernatant was collected, and an IgG concentration in the supernatant was measured by size exclusion chromatography (SEC) (used column: TSK-GEL G3000SWXL, particle size: 5 μm, manufacture by TOSOH Corporation, measuring apparatus: high performance liquid chromatography LC20A, manufactured by Shimadzu Corporation), thereby calculating a formation rate of the IgG/polyE complex. The results are shown in FIG. 9 and Table 7.

The mixed solutions prepared in Test Example 9 are shown in Table 7.

TABLE 7

| | Mixed Solution | | | | | | | |
|---|---|---|---|---|---|---|---|---|
| | Solution A | | | | | Solution B | | |
| | | | | PEG | | Protein for | | Complex |
| | | Polyamino acid | | (4 kDa) | | medical use | | formation |
| | | | polyE | | % | Human IgG | | rate |
| | No. | No. | (mg/mL) | No. | (w/v) | (mg/mL) | pH | (%) |
| Example 9-1 | A22 | e2 | 0.5 | p6 | 6 | 2.0 | 6.0 | 71.7 |
| Example 9-2 | A23 | e2 | 0.5 | p7 | 12 | 2.0 | 6.0 | 86.4 |
| Example 9-3 | A24 | e2 | 0.5 | p8 | 15 | 2.0 | 6.0 | 96.4 |
| Comparative | A′ 3 | e2 | 0.5 | — | — | 2.0 | 6.0 | 7.4 |
| Example 9 | | | | | | | | |

polyE: polyglutamic acid (50 kDa to 100 kDa)
PEG: polyethylene glycol

### (Discussion)

As shown in FIG. 9 and Table 7, it can be appreciated that when a pH of the mixed solution was 6.0, and poly-L-glutamic acid having a long chain (50 kDa to 100 kDa) was used, a formation rate of the complex in which PEG was not added was 10% or less.

On the other hand, it can be appreciated that a formation rate of the complex was recovered up to 70% or more by adding PEG (4 kDa).

### Test Example 10

### Examples 10-1 to 10-3

### (Preparation of Solutions p9 to p11 Containing Polyethylene Glycol)

In a 10 mM citrate buffer solution (pH 6.0), polyethylene glycol (PEG) (20000 Da) was prepared so that the PEG in the mixed solution was at a predetermined concentration as shown in Table 8, thereby preparing solutions p9 to p11.

### (Preparation of Solutions A25 to A27)

50 μL of a solution e2 and 50 μL of each of solutions p9 to p11 were mixed, thereby preparing 100 μL of solutions A25 to A27.

### (Formation of Complex)

50 μL of a solution B was mixed with 100 μL of each of the solutions A25 to A27, thereby preparing a mixed solution. Each mixed solution was allowed to stand at 25° C. for 30 minutes, thereby forming an IgG/polyE/PEG complex.

### (Calculation of Formation Rate of Complex)

After the IgG/polyE/PEG complex was formed, centrifugation was performed at 9000×g for 5 minutes, thereby precipitating the complex. A supernatant was collected, and an IgG concentration in the supernatant was measured by size exclusion chromatography (SEC) (used column: TSK-GEL G3000SWXL, particle size: 5 μm, manufacture by TOSOH Corporation, measuring apparatus: high performance liquid chromatography LC20A, manufactured by Shimadzu Corporation), thereby calculating a formation rate of the IgG/polyE/PEG complex. The results are shown in FIG. 10 and Table 8.

### Comparative Example 10

An IgG/polyE complex was formed and a formation rate thereof was calculated in the same manner as that of Comparative Example 9. The results are shown in FIG. 10 and Table 8.

TABLE 8

| | Mixed solution | | | | | | | |
|---|---|---|---|---|---|---|---|---|
| | Solution A | | | | | Solution B | | |
| | | | | PEG | | Protein for | | Complex |
| | | Polyamino acid | | (20 kDa) | | medical use | | formation |
| | | | polyE | | % | Human IgG | | rate |
| | No. | No. | (mg/mL) | No. | (w/v) | (mg/mL) | pH | (%) |
| Example 10-1 | A25 | e2 | 0.5 | p9 | 6 | 2.0 | 6.0 | 83.6 |
| Example 10-2 | A26 | e2 | 0.5 | p10 | 12 | 2.0 | 6.0 | 92.1 |
| Example 10-3 | A27 | e2 | 0.5 | p11 | 15 | 2.0 | 6.0 | 94.7 |
| Comparative | A′ 3 | e2 | 0.5 | — | — | 2.0 | 6.0 | 7.4 |
| Example 10 | | | | | | | | |

polyE: polyglutamic acid (50 kDa to 100 kDa)
PEG: polyethylene glycol

### (Discussion)

As shown in FIG. 10 and Table 8, it can be appreciated that when a pH of the mixed solution was 6.0, and poly-L-glutamic acid having a long chain (50 kDa to 100 kDa) was used, a formation rate of the complex in which PEG was not added was 10% or less.

On the other hand, it can be appreciated that a formation rate of the complex was recovered up to 80% or more by adding PEG (20 kDa).

The present application is based on the Japanese Patent Application No. 2017-142905 filed on Jul. 24, 2017, and the entirety of the contents of the disclosure is incorporated herein by reference.

1. A method for producing a protein for medical use/polyamino acid complex, the method comprising forming the complex in a mixed solution obtained by mixing a solution A containing a polyamino acid and at least one of alcohol (excluding a hydrophilic polymer) or a hydrophilic polymer (excluding a polyamino acid) with a solution B containing a protein for medical use.

2. The method according to claim 1, wherein a pH of the mixed solution is more than 4.5 and 9.0 or less.

3. The method according to claim 1, wherein the polyamino acid is selected from the group consisting of a polyglutamic acid, polylysine, polyarginine, and water-soluble salts thereof.

4. The method according to claim 1, wherein the alcohol is selected from the group consisting of ethanol, methanol, and trifluoroethanol.

5. The method according to claim 1, wherein the hydrophilic polymer is selected from the group consisting of polyethylene glycol, polypropylene glycol, polyvinyl alcohol, a glycolic acid/L-lactic acid copolymer, polyvinyl pyrrolidone, and a hyaluronic acid.

6. The method according to claim 1, wherein a content of the alcohol is 2 mass % or more and 25 mass % or less with respect to a total amount of the mixed solution.

7. The method according to claim 1, wherein a content of the hydrophilic polymer is 2 mass % or more and 15 mass % or less with respect to a total amount of the mixed solution.

8. The method according to claim 1, wherein
the solution A contains the alcohol, and
a ratio of the number of particles of the protein for medical use/polyamino acid complex with a particle size of 5 μm or more and 10 μm or less to the number of particles of the protein for medical use/polyamino acid complex with a particle size of 1 μm or more and less than 5 μm is more than 0.5%.

9. A complex of a protein for medical use, a polyamino acid, and a hydrophilic polymer.

10. The complex according to claim 9, wherein the polyamino acid is selected from the group consisting of a polyglutamic acid, polylysine, and water-soluble salts thereof.

11. The complex according to claim 9 or 10, wherein the hydrophilic polymer is selected from the group consisting of polyethylene glycol, polypropylene glycol, polyvinyl alcohol, a glycolic acid/L-lactic acid copolymer, polyvinyl pyrrolidone, and a hyaluronic acid.
